(19) United States
(12) Patent Application Publication
(10) Pub. No.: US 2023/0100836 A1
(43) Pub. Date: Mar. 30, 2023

(54) METHODS FOR PROCESSING AND ANALYZING EXTRACELLULAR VESICLES

(71) Applicants: Capsugel Italy S.R.L., Milan (IT); Exosomics SpA, Siena (IT)

(72) Inventors: Davide ZOCCO, Milan (IT); Mattia CRISCUOLI, Siena (IT); Ilaria PASSALACQUA, Siena (IT)

(21) Appl. No.: 17/936,020

(22) Filed: Sep. 28, 2022

Related U.S. Application Data
(60) Provisional application No. 63/249,705, filed on Sep. 29, 2021.

(51) Int. Cl. C12Q 1/6881; G01N 33/569

(52) U.S. Cl. CPC C12Q 1/6881; G01N 33/56966

(57) ABSTRACT

The present disclosure provides methods for processing extracellular vesicles in which the extracellular vesicles are not purified, prior to contacting with a fluorescent staining dye or an antibody. By utilizing a centrifugal filter, excess staining dye or antibody can be readily removed prior to analysis of one or more characteristics of the extracellular vesicles. The methods provide rapid and simple processing and analysis, while maintaining a high concentration of extracellular vesicles.

### CROSS-REFERENCE TO RELATED APPLICATIONS

The present application claims benefit of U.S. Provisional Patent Application No. 63/249,705, filed Sep. 29, 2021, the disclosure of which is incorporated by reference herein in its entirety.

### FIELD OF THE INVENTION

The present disclosure provides methods for processing extracellular vesicles in which the extracellular vesicles are not purified, prior to contacting with a fluorescent staining dye or an antibody. By utilizing a centrifugal filter, excess staining dye or antibody can be readily removed prior to analysis of one or more characteristics of the extracellular vesicles. The methods provide rapid and simple processing and analysis, while maintaining a high concentration of extracellular vesicles.

### BACKGROUND OF THE INVENTION

Research into the applications and treatments using exosomes, or extracellular vesicles, for various cancers and other conditions continue to develop. The ability of these 50-150 nm cell-derived vesicles to deliver various cargo, include proteins, lipids and nucleic acids (including siRNA and antisense nucleic acids), has lead to interest in utilizing them for delivery to various different cell types.

In order to isolate and analyze extraceulluar vesicles from a generating cell population and associated debris, various methods have been developed. However, these traditional approaches often result in loss of extracellular vesicles, or significantly dilute the sample, which can lead to inconsistent or compromised analysis.

What is needed is a simple, rapid process that provides for extracellular vesicle separation and analysis, without unwanted dilution. The present invention provides such processes.

### SUMMARY OF THE INVENTION

In some embodiments, provided herein is a method for processing extracellular vesicles, comprising: concentrating extracellular vesicles in a biological fluid; determining a concentration of the extracellular vesicles; contacting the extracellular vesicles with a fluorescent staining dye or an antibody for an extracellular vesicle surface marker; incubating the contacted extracellular vesicles to generate a labeled extracellular vesicle population; passing the contacted extracellular vesicles through a centrifugal filter comprising a 200-750 kD molecular weight cut off, polyethersulfone filter media, to separate the labeled extracellular vesicle population from excess fluorescent staining dye or excess antibody; and recovering the labeled extracellular vesicle population, wherein the extracellular vesicles are not purified prior to the contacting.

In further embodiments, provided herein is a method for analyzing extracellular vesicles, comprising: concentrating extracellular vesicles in a conditioned medium with a tangential flow filter; determining a concentration of the extracellular vesicles; contacting the extracellular vesicles with a fluorescent staining dye or an antibody for a extracellular vesicle surface marker; incubating the contacted extracellular vesicles to generate a labeled extracellular vesicle population; passing the contacted extracellular vesicles through a centrifugal filter comprising a 300 kD molecular weight cut off, polyethersulfone filter media, to separate the labeled extracellular vesicle population from excess fluorescent staining dye or excess antibody; recovering the labeled extracellular vesicle population; and analyzing the recovered, labeled extracellular vesicle population using a flow cytometer for nanoparticle analysis, wherein the extracellular vesicles are not purified via size exclusion chromatography prior to the contacting.

In additional embodiments, provided herein is a method for processing extracellular vesicles, comprising: concentrating extracellular vesicles in a biological fluid; determining a concentration of the extracellular vesicles to be at least 5×10¹⁰ extracellular vesicles/mL; contacting the extracellular vesicles with an antibody for an extracellular vesicle surface marker; incubating the contacted extracellular vesicles to generate a labeled extracellular vesicle population; diluting the labeled extracellular vesicle population by at least a factor of 1:300; and recovering the labeled extracellular vesicle population, wherein the extracellular vesicles are not purified prior to the contacting.

In still further embodiments, provided herein is a method for analyzing extracellular vesicles, comprising: concentrating extracellular vesicles in a conditioned medium with a tangential flow filter; determining a concentration of the extracellular vesicles to be at least 5×10¹⁰ extracellular vesicles/mL; contacting the extracellular vesicles with an antibody for an extracellular vesicle surface marker; incubating the contacted extracellular vesicles to generate a labeled extracellular vesicle population; diluting the labeled extracellular vesicle population by at least a factor of 1:300; recovering the labeled extracellular vesicle population; and analyzing the recovered, labeled extracellular vesicle population using a flow cytometer for nanoparticle analysis, wherein the extracellular vesicles are not purified via size exclusion chromatography prior to the contacting.

In further embodiments, provided herein is a method for processing extracellular vesicles, comprising: concentrating extracellular vesicles in a biological fluid; determining a concentration of the extracellular vesicles to be at least 5×10¹⁰ extracellular vesicles/mL; contacting the extracellular vesicles with an RNA-specific dye; incubating the contacted extracellular vesicles to generate a labeled extracellular vesicle population; diluting the labeled extracellular vesicle population by at least a factor of 1:300; and recovering the labeled extracellular vesicle population, wherein the extracellular vesicles are not purified prior to the contacting.

In additional embodiments, provided herein is a method for analyzing extracellular vesicles, comprising: concentrating extracellular vesicles in a conditioned medium with a tangential flow filter; determining a concentration of the extracellular vesicles to be at least 5×10¹⁰ extracellular vesicles/mL; contacting the extracellular vesicles with a green fluorescent RNA stain or a red fluorescent RNA stain; incubating the contacted extracellular vesicles to generate a labeled extracellular vesicle population; diluting the labeled extracellular vesicle population by at least a factor of 1:300; recovering the labeled extracellular vesicle population; and analyzing the recovered, labeled extracellular vesicle population using a flow cytometer for nanoparticle analysis, wherein the extracellular vesicles are not purified via size exclusion chromatography prior to the contacting.

### BRIEF DESCRIPTION OF THE DRAWINGS

FIGS. 1A-1H show dye removal from EVs following size exclusion chromatography and filtration.

FIG. 2 shows comparison of different molecular weight cut-off filters for fluorescent dye removal.

FIGS. 3A-3B show the effect of NanoSep 300K filtration on EV size distribution.

FIG. 4A shows staining of purified EVs with anti-tetraspanin antibodies (CD9, CD63 and CD81) with removal of excess antibody EVs via filtration and size exclusion chromatography.

FIGS. 4B-4C show staining of conditioned media and in-process samples with anti-tetraspanin antibodies (CD9, CD63 and CD81) with removal of excess antibody with filtration.

FIGS. 5A-5F show Particle Size Distribution, demonstrating antibody labeling of EVs.

FIGS. 6A-6B show the effect of dilution on confirmation of antibody labeling.

FIG. 7A shows the effect of the dilution method on antibody labeling and EV size.

FIGS. 7B-C show the effect of the dilution method in antibody labeling with conditioned media from MSC- and HEK-293293-derived cultures.

FIGS. 8A-8C show the results of RNA staining and dilution.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps.

In embodiments, provided herein is a method for processing extracellular vesicles. The terms “extracellular vesicles” (EVs) and “exosomes” are used interchangeably herein, and refer to submicron-size or nanometer-size, membrane vesicles, generated from cells either under cellular activation or stress. EVs carry nucleic acids, proteins and lipids from their parent cells, and can be engineered to carry desired nucleic acids, including antisense RNA, microRNA (miRNA) or siRNA. EVs are suitably on the order of about 30 nm to about 200 nm in size.

The processing methods described herein are used to separate EVs from a biological fluid, following their production via one or more cell types. As used herein a “biological fluid” refers to a solution that suitably comprises cells, cellular debris, buffers, cell growth media, etc., that is used in the production of EVs.

In embodiments, the methods for processing EVs include concentrating extracellular vesicles in a biological fluid, i.e., concentrating EVs that are present in a biological fluid. Methods of concentrating EVs include for example, passing the EVs through one or more tangential flow filters to concentrate the EVs (i.e., decrease the fluid volume while maintaining the number of EVs in the sample). Prior to concentrating the EVs via one or more tangential flow filters, the EVs can be processed through one or more centrifugation steps, such as 300×g for about 10 minutes, followed by 1200×g for about 20 minutes, followed by 10,000×g for about 30 min. Additional centrifugation steps can also be used. In addition, the speed and duration of centrifugation can be modified, for example, to between about 200×g-500×g for about 5-20 minutes, followed by about 800×g-1500×g for about 10-30 minutes, followed by about 7,000×g-15,000×g for about 20-40 minutes.

As described herein, the concentrating of the EVs is suitably carried out using one or more tangential flow filters. Tangential flow filtration, also known as crossflow filtration, is a filtration system or process where a feed, inlet or input fluid stream passes parallel to a membrane face as one portion passes through, and out of the membrane (permeate flow) while the remainder (retentate flow) passes within the membrane and can be recirculated back to the input, becomes concentrated, can ultimately be passed to storage or for further processing. A tangential flow filter is suitably comprised of a series of hollow fiber membranes (though a single fiber can also be used), into which a solution is fed. The retentate flow passes within the hollow fiber, retaining EVs within the solution inside of the fiber membrane, while excess volume passes through the fiber membrane and out into the permeate flow. This reduces the volume of the total sample, resulting in a concentrating of the EV sample (an increase in number of EVs per volume). Exemplary materials for use in a tangential flow filter include polymers, including but not limited to, poly(ether sulfone), poly(acrylonitrile) and poly(vinylidene difluoride), cellulose esters, and poly(sulfone). Exemplary tangential flow filters include those available from SPECTRUM LABS® or REPLIGEN®, including MICROKROS® and MIDIKROS® filters, and modifications thereof. In embodiments, the material of the tangential flow filter is a modified poly(ether sulfone) (MPES) filter having a molecular weight cut-off of between about 100 kD (100 kilodaltons) to about 750 kD, more suitably a molecular weight cut-off of about 100 kD to about 500 kD, a molecular weight cut-of of about 200 kD to about 400 kD, or a molecular weight cut-off of 100 kD, 200 kD, 300 kD, 400 kD or 500 kD.

The methods of processing further comprise determining a concentration of the extracellular vesicles. Various methods are known in the art for determining the concentration of extracellular vesicles, and include for example, dynamic light scattering, flow cytometry for nanoparticle analysis (nanoscale flow cytometry) (e.g., NanoFCM (Nottingham, UK), and nanoparticle tracking analysis (Nanosight Instruments, Malvern Instruments; ViewSizer, Horiba), etc.

As described herein, suitably the concentration of extracellular vesicles is determined to be at least about 0.5×10¹⁰ extracellular vesicles/mL, prior to continuing the processing methods. More suitably, the concentration of extracellular vesicles is determined to be at least 1×10¹⁰ extracellular vesicles/mL, prior to continuing the processing methods, more suitably at least 0.8×10¹⁰, at least 0.9×10¹⁰, at least 1.1×10¹⁰, at least 1.2×10¹⁰, at least 1.3×10¹⁰, at least 1.4×10¹⁰, or at least 1.5×10¹⁰. As described herein, it has been determined that by achieving a concentration of EVs of about at least 1×10¹⁰, the remainder of the labeling, cleaning/separating/washing elements of the process, and ultimate analysis of the EVs, can be carried out reproducibly and with reduced overall waste.

As described herein, the methods further comprise contacting the extracellular vesicles with a fluorescent staining dye or an antibody for an extracellular vesicle surface marker. The contacted EVs are then incubated to generate a labeled extracellular vesicle population. In suitable embodiments, the EVs are contacted with a fluorescent staining dye that permeates the membrane of the EVs and stains one or more intra-EV molecules. For example, the fluorescent staining dye can be carboxyfluorescein succinimidyl ester (6-Carboxyfluorescein succinimidyl ester; 5(6)-CFDA-SE) (CFSE), a dye that couples, via its succinimidyl group, to intra-EV molecules, notably, to intracellular lysine residues and other amine sources. Additional dyes, including fluorescent staining dyes, that can be used to label the EVs include, for example, ExoBrite™ EV membrane stain (Biotium, Fremont, Calif.), ExoGlow™ EV stain (System Biosciences, Palo Alto, Calif.), as well as membrane dyes such as PKH67 (Sigma Aldrich). Dyes that stain RNA can also be utilized. For example, RNA staining dyes such as SYTO™ RNASelect™ and Quant-iT™ RiboGreen™ Additional dyes are also known in the art and can be used in the described methods as well.

Suitably, the extracellular vesicles are contacted with the fluorescent staining dye and incubated for at least 1 hour at a temperature of about 30° C.-40° C. For example, the EVs can be contacted with the fluorescent staining dye for about 30 minutes to about 2 hours, or about 30 minutes to about 1.5 hours, or about 45 minutes to about 1.5 hours, or about 1 hour to about 1.5 hours, or about 1.5 hours, at a temperature of about 35° C.-40° C., or about 37° C.

In methods in which the EVs are labeled with antibodies, one or more antibodies can be selected for a specific extracellular vesicle surface marker. That is, a surface marker that is expected to be on the surface of EVs, or desired to be on the surface of EVs that contain a wanted cargo (e.g., protein, etc.). In exemplary embodiments, the antibodies are anti-tetraspanin antibodies, that is antibodies that bind to tetraspanin glycoproteins on the surface of the EVs. Tetraspanins are small membrane proteins (200-350 amino acids), which interact laterally with multiple partner proteins and with each other to form the so-called TEMs (tetraspanin-enriched microdomains). Exemplary antibodies, include, but are not limited to, an anti-CD9 antibody, an anti-CD63 antibody, an anti-CD81 antibody, and an anti-IgG1 antibody. Additional antibodies can include an anti-CD151 antibody, an anti-CD82 antibody, an anti-CD53 antibody, an anti-CD37 antibody, etc. Suitably, the EVs are labeled with combinations of such antibodies, such as a combination of an anti-CD9 antibody, an anti-CD63 antibody, an anti-CD81 antibody, and an anti-IgG1 antibody. For example, (CD9+CD63; CD9+CD81; CD81+CD63) and triple (CD9+CD81+CD63) combinations can be used.

Suitably, the extracellular vesicles are contacted with the antibody(ies) and incubated for at least 30 minutes at a temperature of about 30° C.-40° C. For example, the EVs can be contacted with the antibodies for about 30 minutes to about 2 hours, or about 30 minutes to about 1.5 hours, or about 45 minutes to about 1.5 hours, or about 1 hour to about 1.5 hours, or about 1 hour, at a temperature of about 35° C.-40° C., or about 37° C.

Following the labeling, the extracellular vesicles (including both labelled as well as unlabeled EVs) are passed through a centrifugal filter comprising a 200-750 kD molecular weight cut off, polyethersulfone filter media, to separate the labeled extracellular vesicle population from excess fluorescent staining dye or excess antibody. The labeled extracellular vesicle population is then recovered.

As described herein, it has been surprisingly found that by passing the labeled extracellular vesicle population through a polyethersulfone filter with a cutoff of about 200-750 kD molecular weight, labeled EVs are recovered at a very high number without significant loss of EVs, and without significant dilution of the EVs. In suitable embodiments, the contacted extracellular vesicles (labeled with dye or antibodies) are passed through the centrifugal filter for at least 10 minutes at a centrifugal force of at least 10,000×g. Suitably, the molecular weight cutoff of the polyethersulfone filter is about 200-500 kD, about 200-400 kD, or 200 kD, 300 kD, 400 kD or 500 kD. An exemplary 300 kD molecular weight cut-off filter is a NANOSEP® centrifugal filter with OMEGA™ 300K polyethersulfone membrane from PALL® Corporation (Port Washington, N.Y.).

As described herein, it has surprisingly been found that it is not necessary to purify the extracellular vesicles, prior to contacting them with the fluorescent dye or antibodies, to label the EVs. Traditionally, EVs had to be purified from the cellular growth media solution, prior to labeling. However, it has been determined, as described herein, that simply concentrating the EVs (e.g., via centrifugation or tangential flow filtering, or a combination thereof), without purification, leads to an EV sample that can be labeled, then later purified, recovered and analyzed, resulting in a high concentration of EVs for analysis and providing reproducible analytic results of the EV characteristics. As used herein, the term “purify the extracellular vesicles” refers to the use of a size exclusion chromatography column or other filtration media that separates cell components, debris, etc., present in conditioned media, from EVs, resulting in purified EVs. As described herein, such steps and columns are specifically excluded from the processing and analysis methods described herein, as the present methods do not require, and in embodiments specifically exclude, the use of purified EVs.

Various cells can be used to produce extracellular vesicles, as is known in the art. In exemplary embodiments, the EVs are produced from human embryonic kidney (HEK-293) cells (including HEK-293 cells), Human Caucasian colon adenocarcinoma HT-29 cells, or mesenchymal stem cells (MSCs). Additional cells that can be used to prepare the EVs include, but are not limited to, embryonic stem cell-derived cardiovascular progenitor cells, endothelial progenitor cells, immature dendritic cells (DC), etc. In further embodiments, the EVs can produced from various disease cell lines, including various cancer cells lines. In such embodiments, the methods described herein are useful for analyzing disease characteristics present in EVs excreted from various cell types, such as cancer cells.

In exemplary embodiments, the biologic fluid that contains the EVs is a conditioned medium. As used herein “conditioned medium” or “conditioned media” refers to a cell growth media that has been conditioned with one or more growth factors, into which components have been secreted by the cells, and which can include cellular debris (e.g., lipids, proteins and protein aggregates, nucleic acids, etc.), but does not include whole, intact cells. In exemplary embodiments, the conditioned medium comprises HEK-293, HT-29 or MSC cell growth medium, but is suitably serum-free.

Suitably, cells and their product EVs are produced in a bioreactor, prior to use in the methods of processing described herein. The cells can be prepared in any suitable bioreactor (also called reactor herein) including but not limited to stirred tank, airlift, fiber, microfiber, hollow fiber, ceramic matrix, fluidized bed, fixed bed, and/or spouted bed bioreactors. As used herein, “bioreactor” can include a fermenter or fermentation unit, or any other reaction vessel and the terms “bioreactor” and “reactor” are used interchangeably with “fermenter.” The term fermenter or fermentation refers to both microbial and mammalian cultures. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316 L or any other suitable stainless steel) and Inconel, plastics, and/or glass.

In further embodiments, provided herein is a method for analyzing extracellular vesicles. In exemplary embodiments, such methods comprise concentrating extracellular vesicles in a conditioned medium with a tangential flow filter; determining a concentration of the extracellular vesicles; contacting the extracellular vesicles with a fluorescent staining dye or an antibody for a extracellular vesicle surface marker; incubating the contacted extracellular vesicles to generate a labeled extracellular vesicle population; passing the contacted extracellular vesicles through a centrifugal filter comprising a 300 kD molecular weight cut off, polyethersulfone filter media, to separate the labeled extracellular vesicle population from excess fluorescent staining dye or excess antibody; recovering the labeled extracellular vesicle population; and analyzing the recovered, labeled extracellular vesicle population using a flow cytometer for nanoparticle analysis. As described herein, the extracellular vesicles are suitably not purified via size exclusion chromatography prior to the contacting with dye or antibody.

The methods of analysis described herein allow for the determination of one or more of labeling efficiency, extracellular vesicle number, extracellular vesicle concentration, and extracellular vesicle size. Other analytical techniques, beyond the use of a flow cytometer can also be used, including for example, various fluorescent microscopy techniques, liquid chromatography techniques, mass spectrometry, NMR spectroscopy, microfluidic resistive pulse sensing (MRPS), etc. The methods of analysis described herein can suitably be used as part of a manufacturing process for a quality control check of the EVs during production. Such methods allow for the rapid and easy determination if the methods are producing the desired EVs, so that further manufacturing can be continued, or modified as needed, or halted, due to undesired EVs or EV characteristics.

As described herein, in exemplary embodiments, the extracellular vesicles are contacted with the fluorescent staining dye 6-Carboxyfluorescein succinimidyl ester (CFSE), and suitably incubated for at least 1 hour at a temperature of about 30° C.-40° C.

In embodiments where the EVs are contacted with antibodies, suitably one or more of an anti-CD9 antibody, an anti-CD63 antibody, an anti-CD81 antibody, and/or an anti-IgG1 antibody, are utilized. Suitably, the extracellular vesicles are contacted with the antibody and incubated for at least 30 minutes at a temperature of about 30° C.-40° C.

Various cell populations can be utilized to prepare the EVs. As described herein, suitably the EVs are produced from human embryonic kidney (HEK-293) cells, Human Caucasian colon adenocarcinoma HT-29 cells, or mesenchymal stem cells (MSCs). In such embodiments, the conditioned medium comprises HEK-293, HT-29 or MSC cell growth medium, respectively.

Suitably, the concentration of extracellular vesicles in the conditioned medium is determined using a flow cytometer for nanoparticle analysis, and is suitably at least 1×10¹⁰ extracellular vesicles/mL, prior to labeling with the fluorescent dye or antibodies. In exemplary embodiments, the extracellular vesicles are concentrated using a 300 kD molecular weight cut-off tangential flow filter prior to determining the concentration of the EVs.

As described herein, it has been surprisingly found that the contacted extracellular vesicles can be passed through a centrifugal filter (comprising a 300 kD molecular weight cut off, polyethersulfone filter media) for at least 10 minutes at a centrifugal force of at least 10,000×g, to separate the EVs, while still maintaining a high concentration of the EVs for analysis, and without losing a significant number of the EVs during the filtration process.

In still further embodiments, provided herein is a method for processing extracellular vesicles, comprising concentrating extracellular vesicles in a biological fluid, determining a concentration of the extracellular vesicles to be at least 5×10¹⁰ extracellular vesicles/mL, contacting the extracellular vesicles with an antibody for an extracellular vesicle surface marker, incubating the contacted extracellular vesicles to generate a labeled extracellular vesicle population, diluting the labeled extracellular vesicle population by at least a factor of 1:300, and recovering the labeled extracellular vesicle population. As described herein, the extracellular vesicles are not purified prior to the contacting.

In such embodiments, it has been determined that by establishing a concentration of extracellular vesicles that is at least 5×10¹⁰, and then diluting the EVs by at least a factor of 1:300, no filtration is required following the labeling of the EVs with an antibody to recover labeled EVs for analysis.

As described herein, such methods include concentrating extracellular vesicles in a biological fluid. Methods of concentrating EVs are described herein, and include, for example, centrifuging the EVs as well as passing the EVs through one or more tangential flow filters to concentrate the EVs. Suitably, the EVs are passed through a tangential flow filter that is a modified poly(ether sulfone) (MPES) filter having a molecular weight cut-off of between about 100 kD (100 kilodaltons) to about 400 kD, more suitably a molecular weight cut-off of about 300 kD.

Methods for determining the concentration of extracellular vesicles are described herein, and in suitable embodiments, the EV concentration is determined using a flow cytometer for nanoparticle analysis. The EV concentration is suitably at least about 1×10¹⁰ EVs/mL, more suitably at least about 2×10¹⁰ EVs/mL, at least about 3×10¹⁰ EVs/mL, at least about 4×10¹⁰ EVs/mL, at least about 5×10¹⁰ EVs/mL, at least about 6×10¹⁰ EVs/mL, at least about 7×10¹⁰ EVs/mL, at least about 8×10¹⁰ EVs/mL, at least about 9×10¹⁰ EVs/mL, or at least about 10×10¹⁰ EVs/mL.

Once a concentration of EVs of at least about 5×10¹⁰ EVs/mL is achieved, the EVs can be contacted with antibodies for EV cell surface markers. As described throughout, the antibodies are suitably anti-tetraspanin antibodies, and exemplary antibodies, include, but are not limited to, an anti-CD9 antibody, an anti-CD63 antibody, an anti-CD81 antibody, and an anti-IgG1 antibody.

The number of EVs that are contacted by antibodies is suitably between about 1×10⁷-1×10¹⁰ EVs, more suitably between about 1×10⁸-1×10⁹ EV, or in other embodiments, about 1×10⁸, about 2×10⁸, about 3×10⁸, about 4×10⁸, about 5×10⁸, about 6×10⁸, about 7×10⁸, about 8×10⁸, about 9×10⁸, or about 1×10⁹ EVs are contacted with the antibodies. Conditions for contacting the antibodies to the EVs are known in the art, and suitable include incubating the antibodies and EVS for at least 30 minutes at a temperature of about 30° C.-40° C., suitably for at least 40 minutes, at least 50 minutes, at least 1 hour, at least 1.5 hours or at least 2 hours, and at a temperature of about 35° C.-42° C. or about 37° C. In embodiments, the incubation occurs in a stirred incubator, for example at a rotation speed of about 1,200-1,500 rpm, or about 1,400 rpm.

Following the incubation to label to EVs with the antibodies, the EVs are suitably diluted to at least about 1:200 (vol:vol) using a suitable buffer, such as phosphate buffered saline (PBS), prior to recovery and potential analysis of the EVs, as described herein. In further embodiments, the EVs are diluted to at least about 1:250, 1:300, 1:350, 1:400, 1:450. 1:500, 1:550; 1:600; 1:650; 1:700; 1:750; 1:800; 1:850; 1:900; 1:950; or to at least about 1:1000 (vol:vol), prior to further analysis of the EVs.

As described throughout, various cell types can be utilized to produce the EVs, and in suitable embodiments, the extracellular vesicles are produced from human embryonic kidney (HEK-293) cells, Human Caucasian colon adenocarcinoma HT-29 cells, or mesenchymal stem cells (MSCs). In such embodiments, the biologic fluid is suitably a conditioned medium comprising an HEK-293 or MSC cell growth medium.

In still further embodiments, provided herein is a method for analyzing extracellular vesicles, comprising concentrating extracellular vesicles in a conditioned medium with a tangential flow filter, determining a concentration of the extracellular vesicles to be at least 5×10¹⁰ extracellular vesicles/mL, contacting the extracellular vesicles with an antibody for an extracellular vesicle surface marker, incubating the contacted extracellular vesicles to generate a labeled extracellular vesicle population, diluting the labeled extracellular vesicle population by at least a factor of 1:300; recovering the labeled extracellular vesicle population; and analyzing the recovered, labeled extracellular vesicle population using a flow cytometer for nanoparticle analysis, wherein the extracellular vesicles are not purified via size exclusion chromatography prior to the contacting.

Exemplary antibodies for use in labeling the EVs, including an anti-CD9 antibody, an anti-CD63 antibody, an anti-CD81 antibody, and/or an anti-IgG1 antibody, are described throughout. Suitably, the extracellular vesicles are contacted with between 1×10⁸-1×10⁹ antibody particles, and incubated for at least 30 minutes at a temperature of about 30° C.-40° C.

In embodiments, the extracellular vesicles are produced from human embryonic kidney (HEK-293) cells, Human Caucasian colon adenocarcinoma HT-29 cells, or mesenchymal stem cells (MSCs), and suitably the conditioned medium comprises an HEK-293 or MSC cell growth medium, and the extracellular vesicles are concentrated using a 300 kD molecular weight cut-off tangential flow filter. Methods of determining the concentration of extracellular vesicles, including the use of a flow cytometer for nanoparticle analysis, are described herein.

Various methods of analyzing recovered EVs are described herein, including analysis to determine one or more of labeling efficiency, extracellular vesicle number, extracellular vesicle concentration, and extracellular vesicle size.

In still further embodiments, provided herein is a method for processing extracellular vesicles, comprising concentrating extracellular vesicles in a biological fluid; determining a concentration of the extracellular vesicles to be at least 5×10¹⁰ extracellular vesicles/mL; contacting the extracellular vesicles with an RNA-specific dye; incubating the contacted extracellular vesicles to generate a labeled extracellular vesicle population; diluting the labeled extracellular vesicle population by at least a factor of 1:300; and recovering the labeled extracellular vesicle population, wherein the extracellular vesicles are not purified prior to the contacting in c.

In embodiments, the extracellular vesicles are contacted with a green fluorescent RNA stain or a red fluorescent RNA stain. Suitably RNA stains include, for example, SYTO™ RNASelect™ and Quant-iT™ RiboGreen™ (ThermoFisher, Waltham, Mass.). In exemplary embodiments, the extracellular vesicles are contacted with the RNA-specific dye and incubated for at least 20 minutes at a temperature of about 30° C.-40° C.

As described herein, suitably the extracellular vesicles are produced from human embryonic kidney (HEK-293) cells, Human Caucasian colon adenocarcinoma HT-29 cells, or mesenchymal stem cells (MSCs). In embodiments, the concentration of extracellular vesicles is determined using a flow cytometer for nanoparticle analysis. As described herein, suitably the biologic fluid is conditioned medium comprising an HEK-293, HT-29, or MSC cell growth medium, and the concentrating comprises passing the biological fluid through a tangential flow filter.

In further embodiments, provided herein is a method for analyzing extracellular vesicles, comprising: concentrating extracellular vesicles in a conditioned medium with a tangential flow filter; determining a concentration of the extracellular vesicles to be at least 5×10¹⁰ extracellular vesicles/mL; contacting the extracellular vesicles with a green fluorescent RNA stain or a red fluorescent RNA stain; incubating the contacted extracellular vesicles to generate a labeled extracellular vesicle population; diluting the labeled extracellular vesicle population by at least a factor of 1:300; recovering the labeled extracellular vesicle population; and analyzing the recovered, labeled extracellular vesicle population using a flow cytometer for nanoparticle analysis, wherein the extracellular vesicles are not purified via size exclusion chromatography prior to the contacting in c.

Suitably, the extracellular vesicles are contacted with the green fluorescent RNA stain or the red fluorescent RNA stain (exemplary stains described herein) and incubated for at least 20 minutes at a temperature of about 30° C.-40° C. Suitably, the extracellular vesicles are produced from human embryonic kidney (HEK-293) cells, Human Caucasian colon adenocarcinoma HT-29 cells, or mesenchymal stem cells (MSCs). In embodiments, the concentration of extracellular vesicles is determined using a flow cytometer for nanoparticle analysis. Suitably, the conditioned medium comprises an HEK-293, HT-29, or MSC cell growth medium, and the extracellular vesicles are concentrated using a 300 kD molecular weight cut-off tangential flow filter in b.

As described herein, the recovered, labeled extracellular vesicle population is suitably analyzed to determine one or more of labeling efficiency, extracellular vesicle number, extracellular vesicle concentration, and extracellular vesicle size, for example, as part of a quality control step of a manufacturing process.

### EXAMPLES

### Example 1: Fluorescent and Antibody Labeling and Processing Extracellular Vesicles for Analysis Using NanoSep Filtration

### Materials and Methods

### For CFSE Staining:

| Reagent ID | Description | Supplier | Cat. Number | Storage and handling |
|---|---|---|---|---|
| QC/counting | The Quality Control | NanoFCM | QS2503 | Store at 2-8° C. in the original |
| beads NanoFCM | Nanospheres are designed | | | tube. Protect from light. Do not |
| | for use in the alignment | | | freeze. Vortex vigorously before |
| | of Flow Nano-Analyzer | | | use. Bath sonicate if needed. |
| | and as counting calibrator | | | Dilute with distilled water or |
| | | | | PBS buffer (w/o NaCl). |
| NanoFCMTM | The NanoFCM ™ Silica | NanoFCM | S16M-Exo | Store at 2-8° C in the original |
| Silica | Nanospheres Cocktail #1 | | | tube. To be vortexed before use, |
| Nanospheres | contains a mixture of | | | or bath sonicated. Dilute with |
| Cocktail #1 | silica nanospheres. This | | | distilled water of PBS buffer. |
| | cocktail is specialized for | | | |
| | size distribution analysis | | | |
| | of exosomes. | | | |
| CellTrace ™ CFSE | 5(6)-CFDA-SE | Life | C34554 | Lyophilized powder, dissolved |
| | | Technologies | | in DMSO to be stored in freezer |
| | | Italia Fit. | | (−5-30° C.). Protect from |
| | | | | light. |
| qEVoriginal/35 nm | Size exclusion | Izon | qEVoriginal/35 nm | Store at room temperature |
| | chromatography | | series 1001293 | |
| 1x PBS | Working buffer for | 10X PBS Roche | 11666789001 | |
| | sample dilution | | | |

### For Antibody Labeling

| Reagent ID | Description \| | Supplier | Cat. Number | Storage and handling |
|---|---|---|---|---|
| Anti-CD9 | Anti-human PE | Exbio | 1P-208-T100 | Store at 2-8° C. in the original |
| antibody | | | | tube. Protect from light. Do not |
| | | | | freeze |
| Anti-CD63 | Anti-human PE | Exbio | 1P-343-T100 | Store at 2-8° C. in the original |
| antibody | | | | tube. Protect from light. Do not |
| | | | | freeze |
| Anti-CD81 | Anti-human PE | Exbio | 1P-5S8-T100 | Store at 2-8° C. in the original |
| antibody | | | | tube. Protect from light. Do not |
| | | | | freeze |
| Anti-IgG1 | Anti-human isotype PE | Exbio | 1P-632-C100 | Store at 2-8° C. in the original |
| antibody | | | | tube. Protect from light. Do not |
| | | | | freeze |
| QC/counting | The Quality Control | NanoFCM | QS2503 | Store at 2-8° C. in the original |
| beads | Nanospheres are designed | | | tube. Protect from light. Do not |
| NanoFCM | for use in the alignment | | | freeze. Vortex vigorously before |
| | of Flow Nano-Analyzer | | | use. Bath sonicate if needed. |
| | and as counting calibrator | | | Dilutewith distilled water or |
| | | | | PBS buffer (w/o NaCl). |
| NanoFCMTM | The Nano FCM ™ Silica | NanoFCM | S16M-Exo | Store at 2-8° C in the original |
| Silica | Nanospheres Cocktail #1 | | | tube. To be vortexed before use, |
| Nanospheres | contains a mixture of | | | or bath sonicated. Dilute with |
| Cocktail #1 | silica nanospheres. This | | | distilled water of PBS (1X?) |
| | cocktail is specialized for | | | buffer. |
| | size distribution analysis | | | |
| | of exosomes. | | | |
| 10X PBS | Working buffer (PBS 1X) | Roche | 11666789001 | |
| | for sample dilution | | | |

This procedure describes the quantification and staining of particles with CFSE, or the labeling with anti-tetraspanin antibodies, followed by filtration, for further downstream analysis by a NanoAnalyzerNanoAnalyzer from NanoFCM.

Conditioned media containing extracellular vesicles produced from MSC and HEK-293 cells was concentrated with a MicroKros 300K tangential flow filter in order to reach greater than 5×10¹⁰ (suitably 1×10¹¹ particles/mL). The concentration of EVs was determined using a NanoAnalyzer from NanoFCM.

For comparison, EVs were also purified from MSC and HEK-293 cells using size exclusion chromatography by using Izon (Izon SEC qEV10 35 nm, Cat n° qEV10/35 nm) columns, following the manufacturer's instructions.

Fluorescent Staining Procedure

| Parameters | |
|---|---|
| CFSE: final dilution | 10 μM |
| Optimal part/reaction: | 5E+08-2E+09 total particle |
| Volume of particles | 475 μl |
| Volume of CFSE 200 μM | 25 μl |
| Particles concentration of samples | >1E+9 part/ml |
| Reaction volume | 500 μl |
| Reaction time: | 1 hour 30 minutes |
| Reaction temperature | 37° C. under shaking 1400 |
| | rpm in the dark |
| Diluent used | PBS |
| Extra dye removal | SEC/NanoSep |

475 μl of sample*+ of CFSE 200 μM (up to 10 μl)

Vortex the staining reaction and incubate for 1 h in a thermomixer at 37° C., under shaking conditions in the dark.

After the incubation, remove the excess of CFSE by filtering through a centrifugal filter comprising a 300 kD molecular weight cut off, polyethersulfone filter media (NANOSEP® 300 k) following the manufacturer's instruction and measure the pooled fraction with NanoAnalyzer (NanoFCM). For comparison, a second sample was filtered through a size exclusion column.

Additional separation filters were also examined, as shown in the Results, to determine the best filtration media.

### Antibody Labeling Procedure

Parameters

| | |
|---|---|
| CD9 PE Ab dilution/reaction | 1:10 |
| CD63 PE Ab dilution/reaction | 1:10 |
| CD81 PE Ab dilution/reaction | 1:10 |
| Isotype PE IgG1 Ab | 1:30 |
| Tested Ab dilution range: | 1:1-1:2000 |
| Optimal particles/reaction: | 1E09-1E08 total particle |
| Volume of particles | 7-9 μl |
| Volume of each Ab | 1 μl |
| Particles concentration of samples | >4E+10 part/ml |
| Reaction volume | 10 μl |
| Reaction time: | 1 hour |
| Reaction temperature | 37° C. under shaking 1400 |
| | rpm in the dark |
| Diluent used | PBS 1X |
| Extra dye removal | Dilution |
| Sample dilution for NanoFCM measurement | ≥1:300 |

Measure sample with NanoAnalyzer to check that particles concentration is >4E+9. Exemplary dilutions prior to staining.

EVs from MSC: 1 to 300

EVs from HEK-293: 1 to 500

Prepare reaction tubes as following:

Single staining Ab: 9 μl of sample+1 μl of Ab*

Unstained control: 9 μl of sample+1 μl of PBS 1×

Ab MIX: 1 μl of each Ab*+7-9 μl of samples (up to 10 μl)

* prepare an intermediate dilution when required, i.e. isotype Ab

Vortex the staining reaction and incubate in a thermomixer at 37° C., under shaking in the dark, for 1 h.

When incubation time is over, dilute samples at least 1:300 (or apply higher dilution when needed) and measured with NanoAnalyzer.

### Results and Discussion

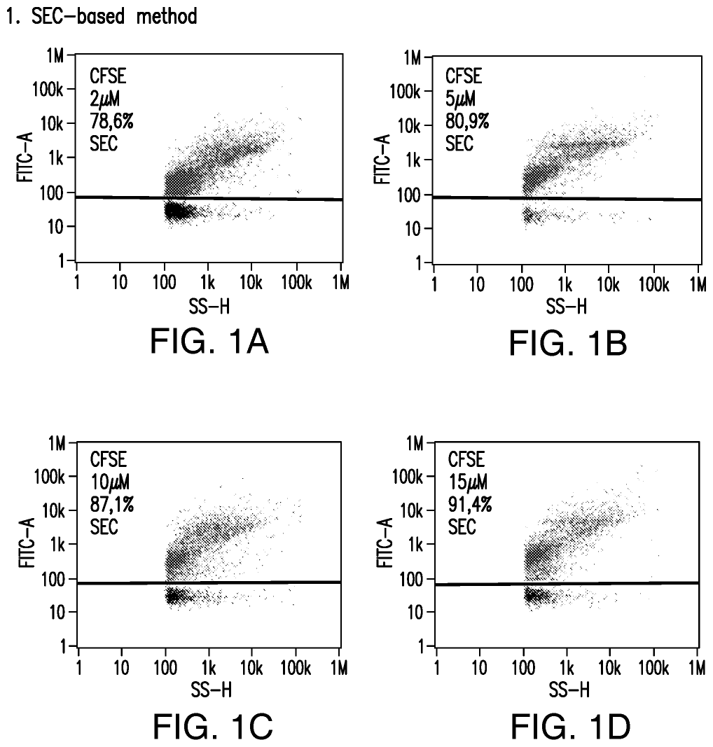

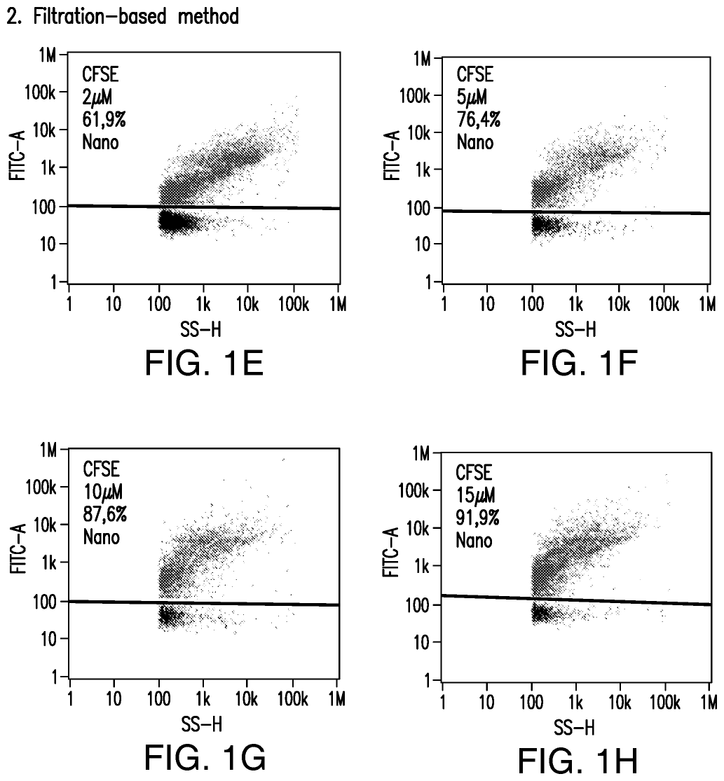

FIGS. 1A-1H show the results of dye removal using size exclusion chromatography (FIGS. 1A-1D) and NanoSep filtration (FIGS. 1E-1H). As indicated, both methods provided comparable staining efficiency and removal of excess fluorescent dye, but the filtration-based method significantly reduced the time for separation and provided for an overall increase in EV concentration, relative to SEC-based methods.

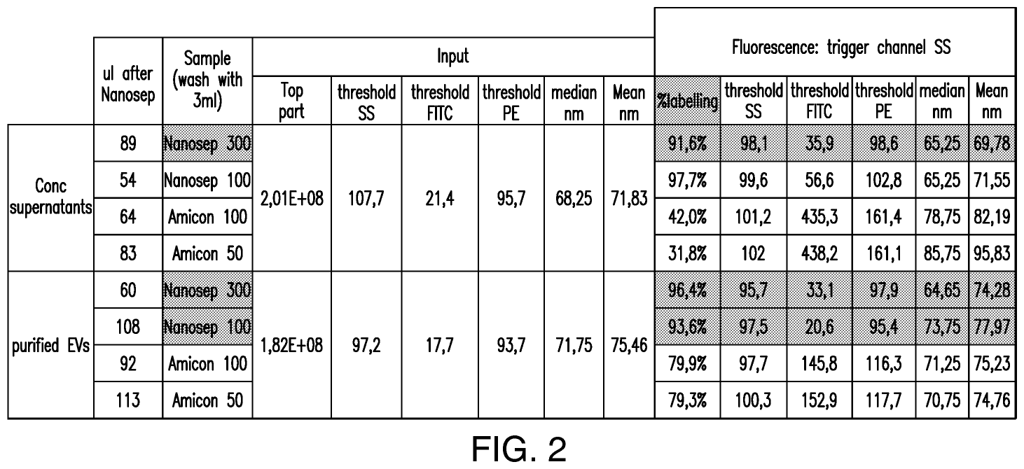

FIG. 2 shows the results comparing different molecular weight cut-off (300 K, 100 K and 50 K) filters at removing CFSE dye from labeled EVs. FIG. 2 also compared the filtration results using unpurified, concentrated EVs (conc supernatants) relative to purified EVs (filtered through size exclusion chromatography column prior to labeling). As indicated the 300 kD molecular weight cut off, polyethersulfone filter media (NANOSEP® 300 k), showed the best results for removing excess CFSE from both conditioned media (conc supernatants) as well as purified EVs. NanoSep 100 K filters effectively removed fluorescent dye from purified EVs, but not from conditioned media.

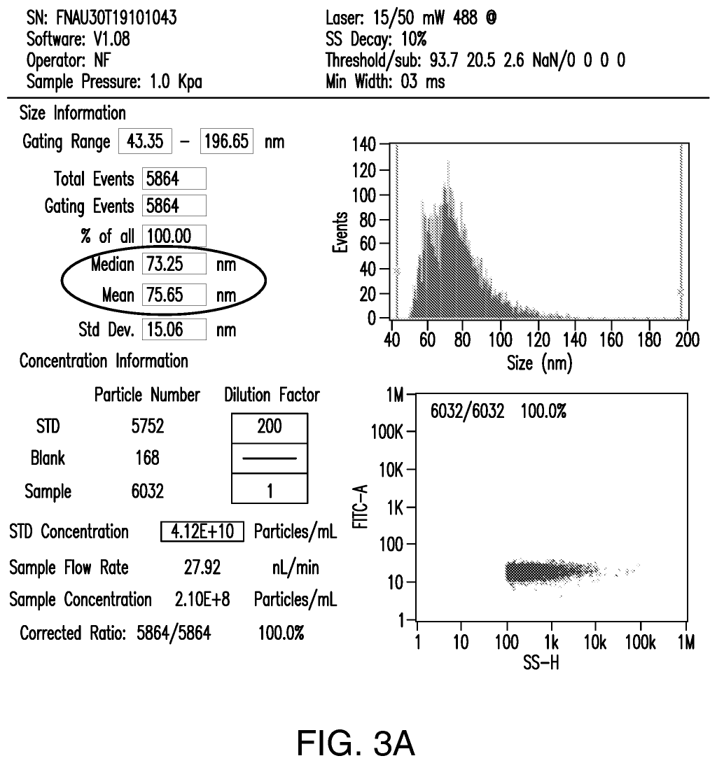

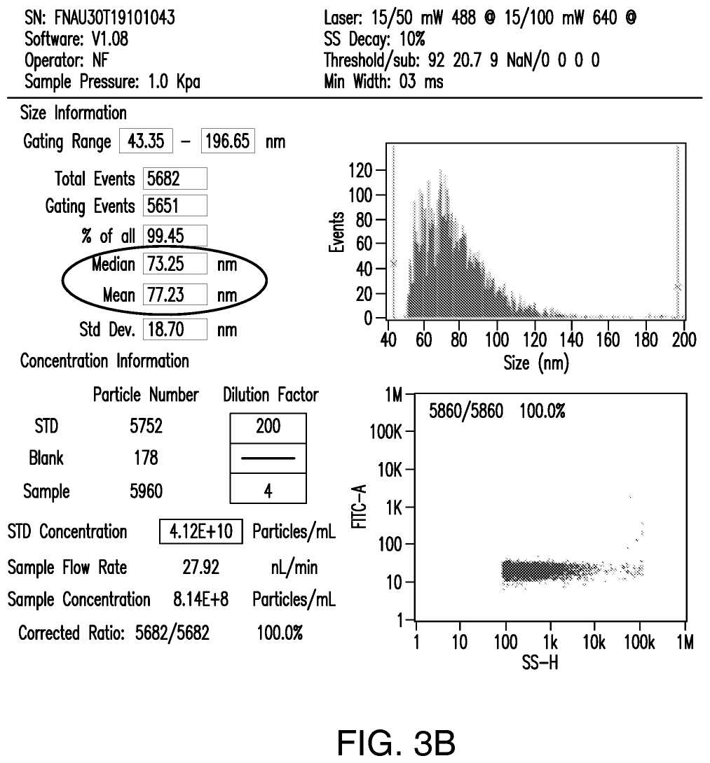

Experiments were also undertaken to confirm that the NanoSep 300K filters do not modify the size distribution of the EVs. The size of SEC purified EVs were measured (FIG. 3A), then passed through a NANOSEP® 300K filter and measured again (FIG. 3B). As shown, the median and mean size of the EVs did not vary significantly.

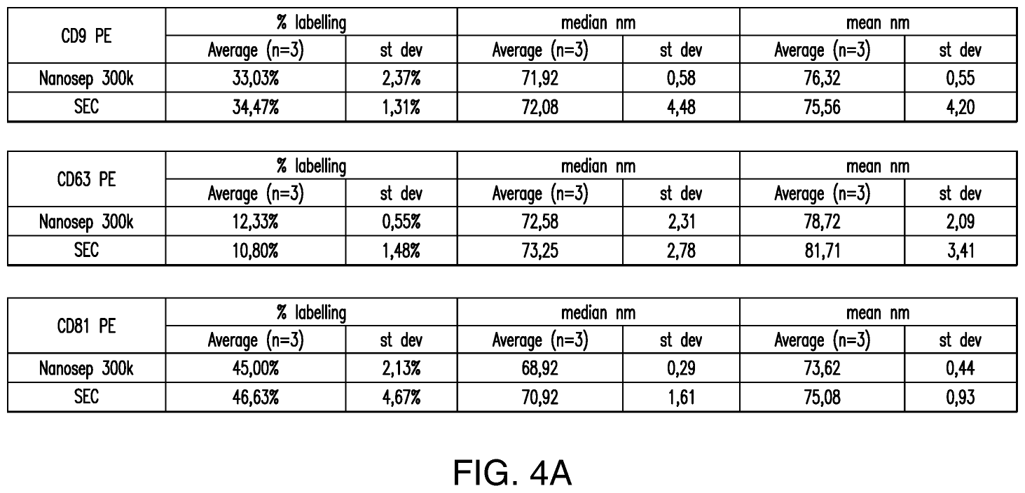

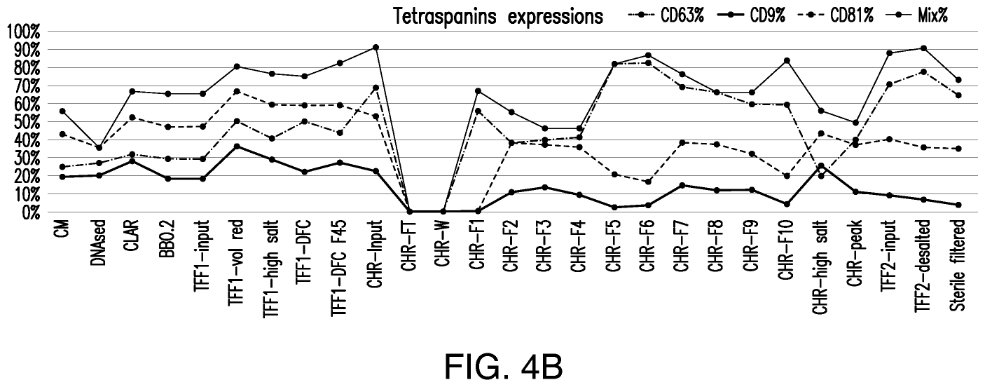

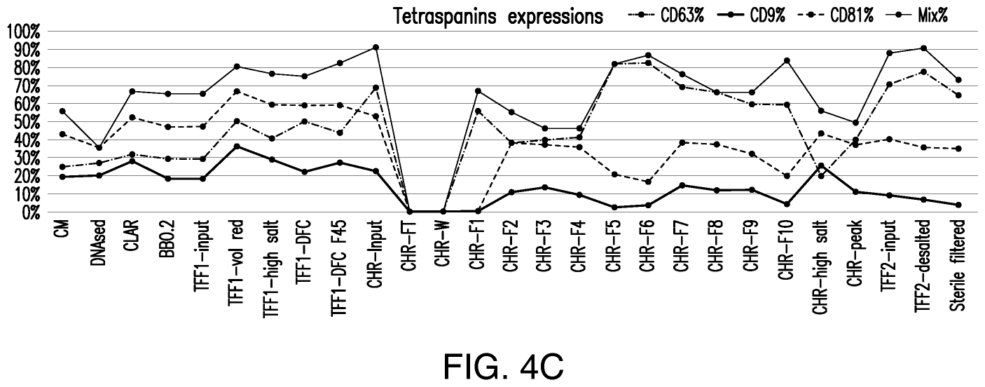

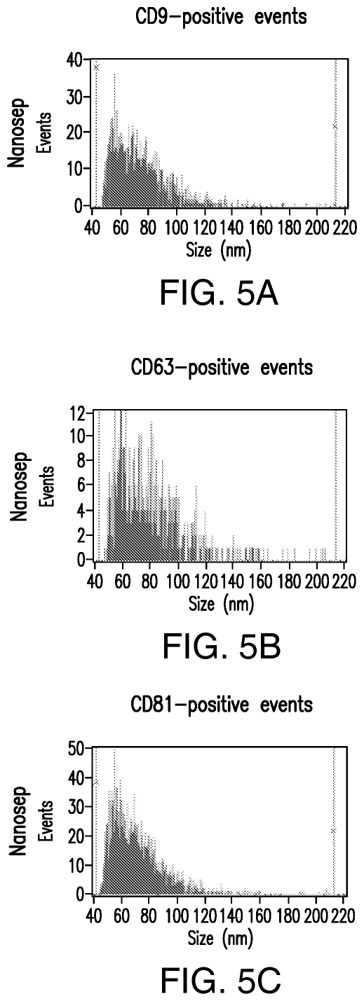

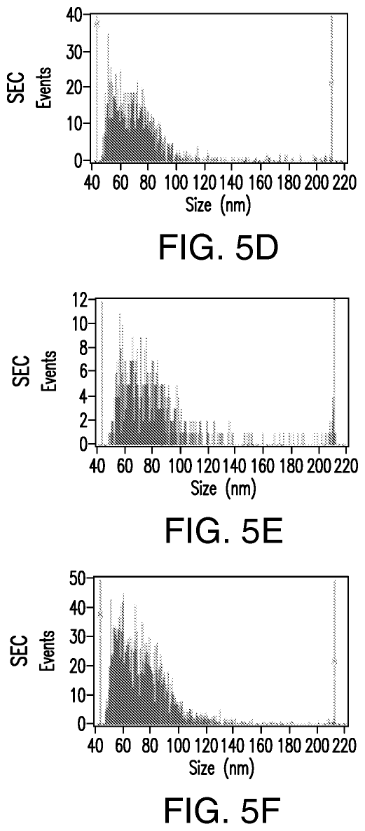

FIG. 4A shows the effective removal of excess antibody from EVs using the NANOSEP® 300K filter, in comparison to the traditional SEC methods. As noted, for all three antibodies, the % labeling of the EVs was comparable using both filtration methods, demonstrating that NanoSep 300K filtration is an effective way to remove excess antibodies, while still maintaining a high concentration of EVs. FIGS. 4B-4C show staining of EVs with anti-tetraspanin antibodies (CD9, CD63 and CD81) with effective removal of excess labeled antibodies from conditioned media and in-process samples of HEK-293 cells (FIG. 4B) and MSCs (FIG. 4C) cell cultures. Particularly, the method was applicable on raw conditioned media (CM) and samples from every step of the EV purification process including post-DNAse treatment (DNased), post-clarification (CLAR), post-bioburden filtration with 0.2 μm cut-off filter (BB0.2), pre-tangential flow filtration 1 (TFF1-input), post-volume reduction (TFF1-vol red), post-high salt wash (TFF1-high salt), post-diafiltration wash (TFF1-DFC), post filtration with 0.45 μm cut-off of diafiltrated wash (TFF1-DFC F45), pre-anion exchange chromatography (CHR input), anion exchange chromatography flow through (CHR-FT), anion exchange chromatography wash (CHR-W), anion exchange chromatography fractions (CHR-FX and peak), anion exchange chromatography high salt wash (CHR-high salt), pre-tangential flow filtration 2 (TFF2-input), post-desalted wash (TFF2-desalted) and post final sterile filtration with a 0.2 μm cut-off filter. FIGS. 5A-5F shows similar results when using Particle Size Distribution as a means to determine effective and unbiased antibody binding.

### Example 2: Antibody Labeling and Processing Extracellular Vesicles for Analysis Without Filtration

Conditioned media from MSC and HEK-293 cells comprising EVs was concentrated using tangential flow filtration (300 kD tangential flow filter). Nanoparticle concentration was determined using a NanoFCM, as greater than 5×10¹⁰ EVs/mL, prior to beginning the antibody labeling process.

Anti-CD9, anti-CD63, and anti-CD81 antibodies were added at between about 2×10⁸-1×10⁹ antibody particles, and incubated for 1 hour at 37° C., with stirring at about 1400 rpm.

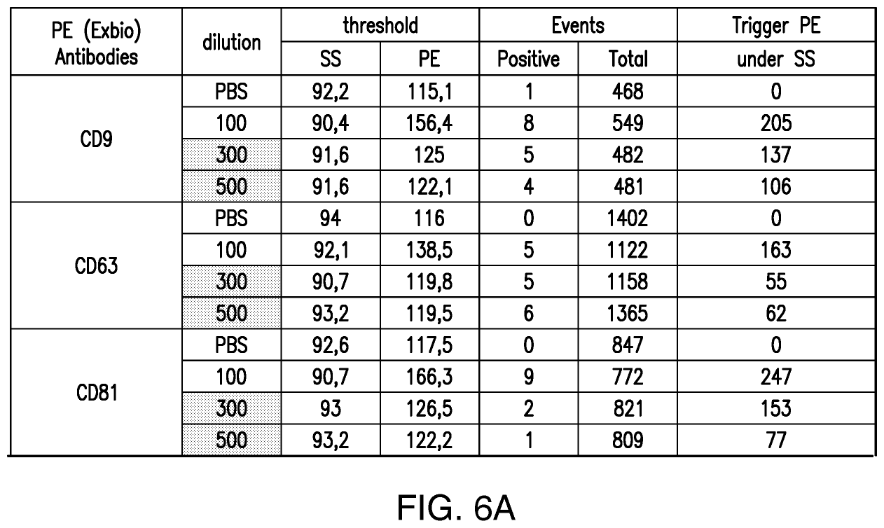

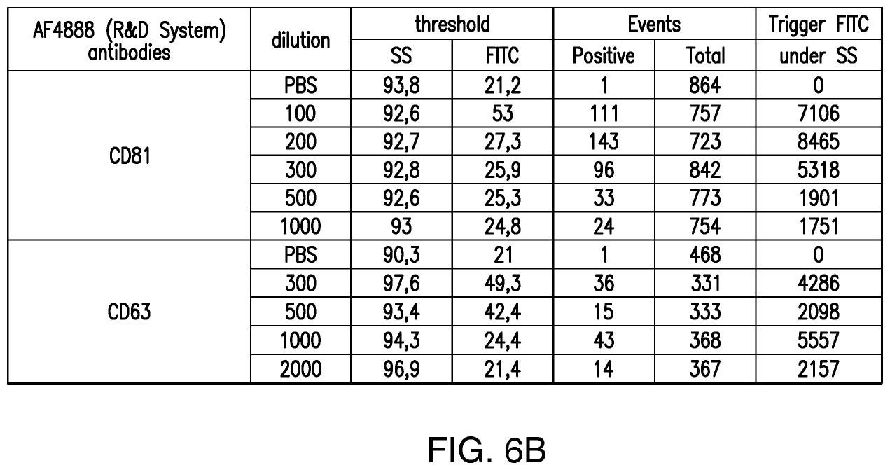

The sample was diluted to greater than 1:300 with appropriate buffer, and then analyzed with a NanoFCM for antibody labeling. FIGS. 6A-6B show that using a dilution factor of greater than 1:300 still allowed for acceptable fluorescence thresholds when measuring antibody labeling.

No filtration was used prior to the analysis, thereby eliminating any concern of removing EVs from the same during filtration.

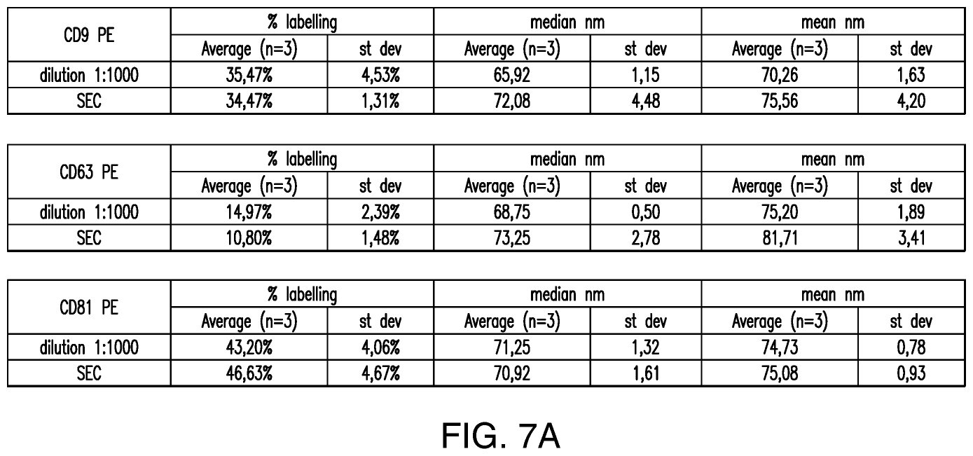

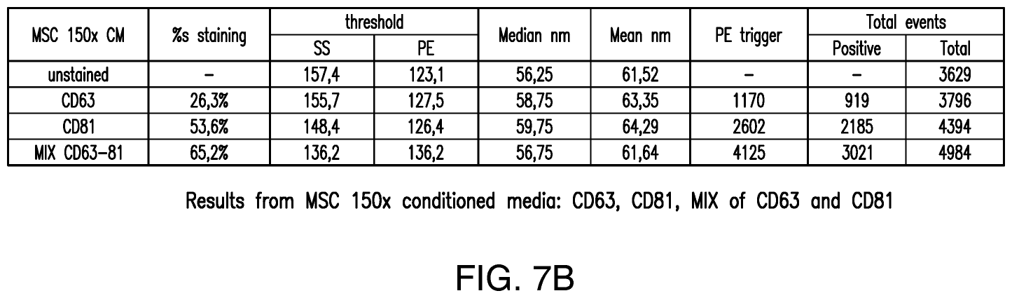

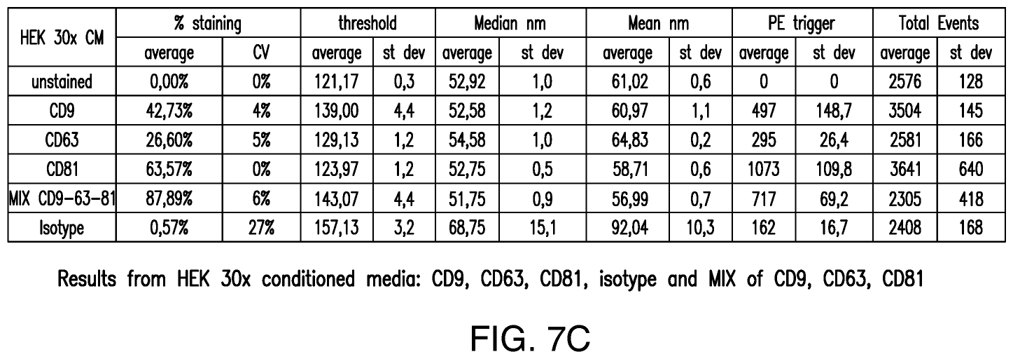

FIG. 7A shows a comparison of the dilution method for antibody staining and analysis as described herein, against a traditional SEC filtration method. As indicated, diluting 1:1000 prior to analysis resulted in a labeling efficiency comparable to that achieved via SEC filtration.

FIG. 7B shows the results of the dilution method for antibody staining and analysis described herein, utilizing concentrated (150×) conditioned media from MSC cultures. Dilution 1:1000 prior to analysis resulted in efficient labelling with % staining, median and mean size (nm) with acceptable threshold values (<200) and number of events (>2000). FIG. 7C shows the results from concentrated (30×) conditioned media from HEK-293 cell cultures. Dilution 1:1000 prior to analysis resulted in efficient labelling with % staining, median and mean size (nm) with acceptable threshold values (<200) and number of events (>2000).

### Example 3: RNA Staining and Processing Extracellular Vesicles for Analysis Without Filtration

Conditioned media from MSC and HEK-293 cells comprising EVs was concentrated using tangential flow filtration (300 kD tangential flow filter). Nanoparticle concentration was determined using NanoAnalyzer (NanoFCM), as greater than 5×10¹⁰ EVs/mL, prior to beginning the RNA staining process.

SYTO™ RNASelect™ (Syto; Thermo Fisher Scientific) and Quant-iT™ RiboGreen® (RiboGreen; Thermo Fisher Scientific) were added at 2504 and diluted 1:50, respectively. For Syto and RiboGreen, samples were incubated for 20 minutes and 30 minutes, respectively, both at 37° C. under shaking, protected from light. The sample was diluted to greater than 1:300 with appropriate buffer, and then analyzed with a NanoFCM for antibody labeling. A second sample was passed through a NanoSep 300K filter to compare filtration of the dye vs. dilution.

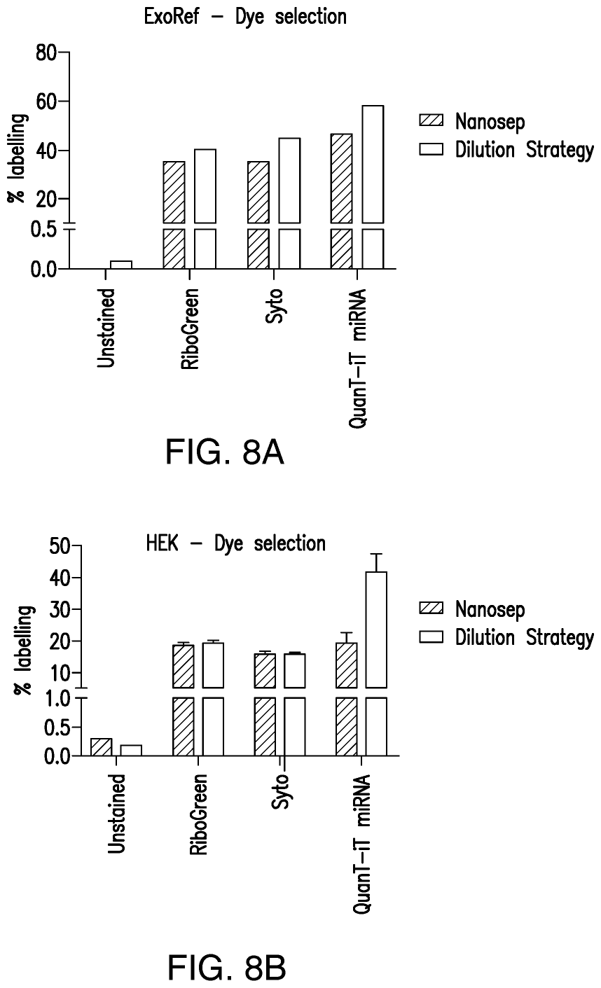

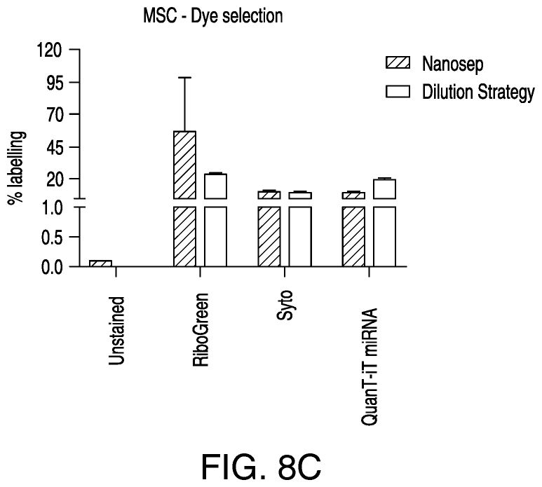

FIG. 8A shows that both RiboGreen and Syto effectively label an Exosome reference sample (ExoRef). For all graphs (8A-8C) QuantT-iT miRNA is shown as a control for RNA labeling.

FIG. 8B shows EVs prepared from HEK-293 cells are effectively labeled with both RiboGreen and Syto, and that the use of dilution results in approximately the same determined labeling efficiency, when compared with the Nanosep filtration.

FIG. 8C shows EVs prepared from MSC cells are effectively labeled with both RiboGreen and Syto, and that the use of dilution results in approximately the same determined labeling efficiency, when compared with the Nanosep filtration.

These results demonstrate that dilution can be effectively used to characterize EVs labeled with RNA stains, and that filtration is not needed to separate non-bound fluorescent RNA stains prior to analysis. This is a surprising and unexpected result, and provides a rapid and simple method to prepare EVs for analysis, as described throughout.

Similar results can be expected, based on the experimental data provided herein, for EVs in conditioned media, where the EVs have not been purified. These methods include the use of tangential flow filtration (300 kD tangential flow filter) to filter excess RNA dyes, as well as dilution methods to remove excess dyes. Both methods are expected to yield similar results to those provided on purified EVs, when translated to EVs in conditioned media.

### Exemplary Embodiments

Embodiment 1 is a method for processing extracellular vesicles, comprising: concentrating extracellular vesicles in a biological fluid; determining a concentration of the extracellular vesicles; contacting the extracellular vesicles with a fluorescent staining dye or an antibody for an extracellular vesicle surface marker; incubating the contacted extracellular vesicles to generate a labeled extracellular vesicle population; passing the contacted extracellular vesicles through a centrifugal filter comprising a 200-750 kD molecular weight cut off, polyethersulfone filter media, to separate the labeled extracellular vesicle population from excess fluorescent staining dye or excess antibody; and recovering the labeled extracellular vesicle population, wherein the extracellular vesicles are not purified prior to the contacting.

Embodiment 2 includes the method of Embodiment 1, wherein the concentrating comprises passing the biological fluid through a tangential flow filter.

Embodiment 3 includes the method of Embodiment 2, wherein the tangential flow filter has a molecular weight cut-off of about 100 kD to about 500 kD.

Embodiment 4 includes the method of Embodiment 1, wherein the extracellular vesicles are contacted with the fluorescent staining dye 6-Carboxyfluorescein succinimidyl ester (CFSE).

Embodiment 5 includes the method of Embodiment 1, wherein the extracellular vesicles are contacted with an anti-CD9 antibody, an anti-CD63 antibody, an anti-CD81 antibody, and/or an anti-IgG1 antibody.

Embodiment 6 includes the method of any of Embodiments 1-4, wherein the extracellular vesicles are contacted with the fluorescent staining dye and incubated for at least 1 hour at a temperature of about 30° C.-40° C.

Embodiment 7 includes the method Embodiments 1-3 or Embodiment 5, wherein the extracellular vesicles are contacted with the antibody and incubated for at least 30 minutes at a temperature of about 30° C.-40° C.

Embodiment 8 includes the method of any of Embodiments 1-7, wherein the extracellular vesicles are produced from human embryonic kidney (HEK-293) cells, Human Caucasian colon adenocarcinoma HT-29 cells, or mesenchymal stem cells (MSCs).

Embodiment 9 includes the method of any of Embodiments 1-8, wherein the concentration of extracellular vesicles in the biological fluid is determined using a flow cytometer for nanoparticle analysis.

Embodiment 10 includes the method of any of Embodiments 1-9, wherein the concentration of extracellular vesicles is determined to be at least 1×10¹⁰ extracellular vesicles/mL, prior to the contacting.

Embodiment 11 includes the method of any of Embodiments 1-10, wherein the biologic fluid is conditioned medium comprising HEK-293, HT-29 or MSC cell growth medium.

Embodiment 12 includes the method of any of Embodiments 1-11, wherein the contacted extracellular vesicles are passed through the centrifugal filter for at least 10 minutes at a centrifugal force of at least 10,000×g.

Embodiment 13 is a method for analyzing extracellular vesicles, comprising: concentrating extracellular vesicles in a conditioned medium with a tangential flow filter; determining a concentration of the extracellular vesicles; contacting the extracellular vesicles with a fluorescent staining dye or an antibody for a extracellular vesicle surface marker; incubating the contacted extracellular vesicles to generate a labeled extracellular vesicle population; passing the contacted extracellular vesicles through a centrifugal filter comprising a 300 kD molecular weight cut off, polyethersulfone filter media, to separate the labeled extracellular vesicle population from excess fluorescent staining dye or excess antibody; recovering the labeled extracellular vesicle population; and analyzing the recovered, labeled extracellular vesicle population using a flow cytometer for nanoparticle analysis, wherein the extracellular vesicles are not purified via size exclusion chromatography prior to the contacting.

Embodiment 14 includes the method of Embodiment 13, wherein the extracellular vesicles are contacted with the fluorescent staining dye 6-Carboxyfluorescein succinimidyl ester (CFSE).

Embodiment 15 includes the method of Embodiment 13, wherein the extracellular vesicles are contacted with an anti-CD9 antibody, an anti-CD63 antibody, an anti-CD81 antibody, and/or an anti-IgG1 antibody.

Embodiment 16 includes the method of Embodiment 13 or Embodiment 14, wherein the extracellular vesicles are contacted with the fluorescent staining dye and incubated for at least 1 hour at a temperature of about 30° C.-40° C.

Embodiment 17 includes the method of Embodiment 13 or Embodiment 15, wherein the extracellular vesicles are contacted with the antibody and incubated for at least 30 minutes at a temperature of about 30° C.-40° C.

Embodiment 18 includes the method of any of Embodiments 13-17, wherein the extracellular vesicles are produced from human embryonic kidney (HEK-293) cells, Human Caucasian colon adenocarcinoma HT-29 cells, or mesenchymal stem cells (MSCs).

Embodiment 19 includes the method of any of Embodiments 13-18, wherein the concentration of extracellular vesicles in the conditioned medium is determined using a flow cytometer for nanoparticle analysis.

Embodiment 20 includes the method of any of Embodiments 13-19, wherein the concentration of extracellular vesicles is determined to be at least 1×10¹⁰ extracellular vesicles/mL, prior to the contacting in c.

Embodiment 21 includes the method of any of Embodiments 13-20, wherein the conditioned medium comprises HEK-293, HT-29, or MSC cell growth medium, and the extracellular vesicles are concentrated using a 300 kD molecular weight cut-off tangential flow filter in b.

Embodiment 22 includes the method of any of Embodiments 13-21, wherein the contacted extracellular vesicles are passed through the centrifugal filter for at least 10 minutes at a centrifugal force of at least 10,000×g.

Embodiment 23 includes the method of any of Embodiments 13-22, wherein the recovered, labeled extracellular vesicle population is analyzed to determine one or more of labeling efficiency, extracellular vesicle number, extracellular vesicle concentration, and extracellular vesicle size.

Embodiment 24 is a method for processing extracellular vesicles, comprising: concentrating extracellular vesicles in a biological fluid; determining a concentration of the extracellular vesicles to be at least 5×10¹⁰ extracellular vesicles/mL; contacting the extracellular vesicles with an antibody for an extracellular vesicle surface marker; incubating the contacted extracellular vesicles to generate a labeled extracellular vesicle population; diluting the labeled extracellular vesicle population by at least a factor of 1:300; and recovering the labeled extracellular vesicle population, wherein the extracellular vesicles are not purified prior to the contacting.

Embodiment 25 includes the method of Embodiment 24, wherein the extracellular vesicles are contacted with an anti-CD9 antibody, an anti-CD63 antibody, an anti-CD81 antibody, and/or an anti-IgG1 antibody.

Embodiment 26 includes the method of Embodiment 24 or Embodiment 25, wherein 1×10⁸-1×10⁹ extracellular vesicles are contacted with antibody particles.

Embodiment 27 includes the method of any of Embodiments 24-26, wherein the extracellular vesicles are contacted with the antibody and incubated for at least 30 minutes at a temperature of about 30° C.-40° C.

Embodiment 28 includes the method of any of Embodiments 24-27, wherein the extracellular vesicles are produced from human embryonic kidney (HEK-293) cells, Human Caucasian colon adenocarcinoma HT-29 cells, or mesenchymal stem cells (MSCs).

Embodiment 29 includes the method of any of Embodiments 24-28, wherein the concentration of extracellular vesicles is determined using a flow cytometer for nanoparticle analysis.

Embodiment 30 includes the method of any of Embodiments 24-29, wherein the biologic fluid is conditioned medium comprising an HEK-293, HT-29, or MSC cell growth medium, and the concentrating comprises passing the biological fluid through a tangential flow filter.

Embodiment 31 is a method for analyzing extracellular vesicles, comprising: concentrating extracellular vesicles in a conditioned medium with a tangential flow filter; determining a concentration of the extracellular vesicles to be at least 5×10¹⁰ extracellular vesicles/mL; contacting the extracellular vesicles with an antibody for an extracellular vesicle surface marker; incubating the contacted extracellular vesicles to generate a labeled extracellular vesicle population; diluting the labeled extracellular vesicle population by at least a factor of 1:300; recovering the labeled extracellular vesicle population; and analyzing the recovered, labeled extracellular vesicle population using a flow cytometer for nanoparticle analysis, wherein the extracellular vesicles are not purified via size exclusion chromatography prior to the contacting.

Embodiment 32 includes the method of Embodiment 31, wherein the extracellular vesicles are contacted with an anti-CD9 antibody, an anti-CD63 antibody, an anti-CD81 antibody, and/or an anti-IgG1 antibody.

Embodiment 33 includes the method of Embodiment 31 or Embodiment 32, wherein the extracellular vesicles are contacted with between 1×10⁸-1×10⁹ antibody particles.

Embodiment 34 includes the method of any of Embodiments 31-33, wherein the extracellular vesicles are contacted with the antibody and incubated for at least 30 minutes at a temperature of about 30° C.-40° C.

Embodiment 35 includes the method of any of Embodiments 31-34, wherein the extracellular vesicles are produced from human embryonic kidney (HEK-293) cells, Human Caucasian colon adenocarcinoma HT-29 cells, or mesenchymal stem cells (MSCs).

Embodiment 36 includes the method of any of Embodiments 31-35, wherein the concentration of extracellular vesicles is determined using a flow cytometer for nanoparticle analysis.

Embodiment 37 includes the method of any of Embodiments 31-36, wherein the conditioned medium comprises an HEK-293, HT-29, or MSC cell growth medium, and the extracellular vesicles are concentrated using a 300 kD molecular weight cut-off tangential flow filter in b.

Embodiment 38 includes the method of any of Embodiments 31-37, wherein the recovered, labeled extracellular vesicle population is analyzed to determine one or more of labeling efficiency, extracellular vesicle number, extracellular vesicle concentration, and extracellular vesicle size.

Embodiment 39 is a method for processing extracellular vesicles, comprising: concentrating extracellular vesicles in a biological fluid; determining a concentration of the extracellular vesicles to be at least 5×10¹⁰ extracellular vesicles/mL; contacting the extracellular vesicles with an RNA-specific dye; incubating the contacted extracellular vesicles to generate a labeled extracellular vesicle population; diluting the labeled extracellular vesicle population by at least a factor of 1:300; and recovering the labeled extracellular vesicle population, wherein the extracellular vesicles are not purified prior to the contacting.

Embodiment 40 includes the method of Embodiment 39, wherein the extracellular vesicles are contacted with a green fluorescent RNA stain or a red fluorescent RNA stain.

Embodiment 41 includes the method of any of Embodiments 39-40, wherein the extracellular vesicles are contacted with the RNA-specific dye and incubated for at least 20 minutes at a temperature of about 30° C.-40° C.

Embodiment 42 includes the method of any of Embodiments 39-41, wherein the extracellular vesicles are produced from human embryonic kidney (HEK-293) cells, Human Caucasian colon adenocarcinoma HT-29 cells, or mesenchymal stem cells (MSCs).

Embodiment 43 includes the method of any of Embodiments 39-42, wherein the concentration of extracellular vesicles is determined using a flow cytometer for nanoparticle analysis.

Embodiment 44 includes the method of any of Embodiments 39-43, wherein the biologic fluid is conditioned medium comprising an HEK-293, HT-29, or MSC cell growth medium, and the concentrating comprises passing the biological fluid through a tangential flow filter.

Embodiment 45 is a method for analyzing extracellular vesicles, comprising: concentrating extracellular vesicles in a conditioned medium with a tangential flow filter; determining a concentration of the extracellular vesicles to be at least 5×10¹⁰ extracellular vesicles/mL; contacting the extracellular vesicles with a green fluorescent RNA stain or a red fluorescent RNA stain; incubating the contacted extracellular vesicles to generate a labeled extracellular vesicle population; diluting the labeled extracellular vesicle population by at least a factor of 1:300; recovering the labeled extracellular vesicle population; and analyzing the recovered, labeled extracellular vesicle population using a flow cytometer for nanoparticle analysis, wherein the extracellular vesicles are not purified via size exclusion chromatography prior to the contacting.

Embodiment 46 includes the method of Embodiment 45, wherein the extracellular vesicles are contacted with the green fluorescent RNA stain or the red fluorescent RNA stain and incubated for at least 20 minutes at a temperature of about 30° C.-40° C.

Embodiment 47 includes the method of any of Embodiments 45-46, wherein the extracellular vesicles are produced from human embryonic kidney (HEK-293) cells, Human Caucasian colon adenocarcinoma HT-29 cells, or mesenchymal stem cells (MSCs).

Embodiment 48 includes the method of any of Embodiments 45-47, wherein the concentration of extracellular vesicles is determined using a flow cytometer for nanoparticle analysis.

Embodiment 49 includes the method of any of Embodiments 45-48, wherein the conditioned medium comprises an HEK-293, HT-29, or MSC cell growth medium, and the extracellular vesicles are concentrated using a 300 kD molecular weight cut-off tangential flow filter in b.

Embodiment 50 includes the method of any of Embodiments 45-49, wherein the recovered, labeled extracellular vesicle population is analyzed to determine one or more of labeling efficiency, extracellular vesicle number, extracellular vesicle concentration, and extracellular vesicle size.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A method for processing extracellular vesicles, comprising:
a. concentrating extracellular vesicles in a biological fluid;
b. determining a concentration of the extracellular vesicles;
c. contacting the extracellular vesicles with a fluorescent staining dye or an antibody for an extracellular vesicle surface marker;
d. incubating the contacted extracellular vesicles to generate a labeled extracellular vesicle population;
e. passing the contacted extracellular vesicles through a centrifugal filter comprising a 200-750 kD molecular weight cut off, polyethersulfone filter media, to separate the labeled extracellular vesicle population from excess fluorescent staining dye or excess antibody; and
f. recovering the labeled extracellular vesicle population,
wherein the extracellular vesicles are not purified prior to the contacting in c.

2. The method of claim 1, wherein the concentrating comprises passing the biological fluid through a tangential flow filter.

3. The method of claim 2, wherein the tangential flow filter has a molecular weight cut-off of about 100 kD to about 500 kD.

4. The method of claim 1, wherein the extracellular vesicles are contacted with the fluorescent staining dye 6-Carboxyfluorescein succinimidyl ester (CFSE).

5. The method of claim 1, wherein the extracellular vesicles are contacted with an anti-CD9 antibody, an anti-CD63 antibody, an anti-CD81 antibody, and/or an anti-IgG1 antibody.

6. The method of claim 1, wherein the extracellular vesicles are contacted with the fluorescent staining dye and incubated for at least 1 hour at a temperature of about 30° C.-40° C.

7. The method of claim 1, wherein the extracellular vesicles are contacted with the antibody and incubated for at least 30 minutes at a temperature of about 30° C.-40° C.

8. The method of claim 1, wherein the extracellular vesicles are produced from human embryonic kidney (HEK-293) cells, Human Caucasian colon adenocarcinoma HT-29 cells, or mesenchymal stem cells (MSCs).

9. The method of claim 1, wherein the concentration of extracellular vesicles in the biological fluid is determined using a flow cytometer for nanoparticle analysis.

10. The method of claim 1, wherein the concentration of extracellular vesicles is determined to be at least 1×10¹⁰ extracellular vesicles/mL, prior to the contacting in c.

11. The method of claim 1, wherein the biologic fluid is conditioned medium comprising HEK-293, HT-29 or MSC cell growth medium.

12. The method of claim 1, wherein the contacted extracellular vesicles are passed through the centrifugal filter for at least 10 minutes at a centrifugal force of at least 10,000×g.

13. A method for analyzing extracellular vesicles, comprising:
a. concentrating extracellular vesicles in a conditioned medium with a tangential flow filter;
b. determining a concentration of the extracellular vesicles;
c. contacting the extracellular vesicles with a fluorescent staining dye or an antibody for a extracellular vesicle surface marker;
d. incubating the contacted extracellular vesicles to generate a labeled extracellular vesicle population;
e. passing the contacted extracellular vesicles through a centrifugal filter comprising a 300 kD molecular weight cut off, polyethersulfone filter media, to separate the labeled extracellular vesicle population from excess fluorescent staining dye or excess antibody;
f. recovering the labeled extracellular vesicle population; and
g. analyzing the recovered, labeled extracellular vesicle population using a flow cytometer for nanoparticle analysis,
wherein the extracellular vesicles are not purified via size exclusion chromatography prior to the contacting in c.

14. The method of claim 13, wherein the extracellular vesicles are contacted with the fluorescent staining dye 6-Carboxyfluorescein succinimidyl ester (CFSE).

15. The method of claim 13, wherein the extracellular vesicles are contacted with an anti-CD9 antibody, an anti-CD63 antibody, an anti-CD81 antibody, and/or an anti-IgG1 antibody.

16. The method of claim 13, wherein the extracellular vesicles are contacted with the fluorescent staining dye and incubated for at least 1 hour at a temperature of about 30° C.-40° C.

17. The method of claim 13, wherein the extracellular vesicles are contacted with the antibody and incubated for at least 30 minutes at a temperature of about 30° C.-40° C.

18. The method of claim 13, wherein the extracellular vesicles are produced from human embryonic kidney (HEK-293) cells, Human Caucasian colon adenocarcinoma HT-29 cells, or mesenchymal stem cells (MSCs).

19. The method of claim 13, wherein the concentration of extracellular vesicles in the conditioned medium is determined using a flow cytometer for nanoparticle analysis.

20. The method of claim 13, wherein the concentration of extracellular vesicles is determined to be at least 1×10¹⁰ extracellular vesicles/mL, prior to the contacting in c.

21. The method of claim 13, wherein the conditioned medium comprises HEK-293, HT-29, or MSC cell growth medium, and the extracellular vesicles are concentrated using a 300 kD molecular weight cut-off tangential flow filter in b.

22. The method of claim 13, wherein the contacted extracellular vesicles are passed through the centrifugal filter for at least 10 minutes at a centrifugal force of at least 10,000×g.

23. The method of claim 13, wherein the recovered, labeled extracellular vesicle population is analyzed to determine one or more of labeling efficiency, extracellular vesicle number, extracellular vesicle concentration, and extracellular vesicle size.

24. A method for processing extracellular vesicles, comprising:
a. concentrating extracellular vesicles in a biological fluid;
b. determining a concentration of the extracellular vesicles to be at least 5×10¹⁰ extracellular vesicles/mL;
c. contacting the extracellular vesicles with an antibody for an extracellular vesicle surface marker;
d. incubating the contacted extracellular vesicles to generate a labeled extracellular vesicle population;
e. diluting the labeled extracellular vesicle population by at least a factor of 1:300; and
f. recovering the labeled extracellular vesicle population,
wherein the extracellular vesicles are not purified prior to the contacting in c.

25. A method for analyzing extracellular vesicles, comprising:
a. concentrating extracellular vesicles in a conditioned medium with a tangential flow filter;
b. determining a concentration of the extracellular vesicles to be at least 5×10¹⁰ extracellular vesicles/mL;
c. contacting the extracellular vesicles with an antibody for an extracellular vesicle surface marker;
d. incubating the contacted extracellular vesicles to generate a labeled extracellular vesicle population;
e. diluting the labeled extracellular vesicle population by at least a factor of 1:300;
f. recovering the labeled extracellular vesicle population; and
g. analyzing the recovered, labeled extracellular vesicle population using a flow cytometer for nanoparticle analysis,
wherein the extracellular vesicles are not purified via size exclusion chromatography prior to the contacting in c.

26. A method for processing extracellular vesicles, comprising:
a. concentrating extracellular vesicles in a biological fluid;
b. determining a concentration of the extracellular vesicles to be at least 5×10¹⁰ extracellular vesicles/mL;
c. contacting the extracellular vesicles with an RNA-specific dye;
d. incubating the contacted extracellular vesicles to generate a labeled extracellular vesicle population;
e. diluting the labeled extracellular vesicle population by at least a factor of 1:300; and
f. recovering the labeled extracellular vesicle population,
wherein the extracellular vesicles are not purified prior to the contacting in c.

27. A method for analyzing extracellular vesicles, comprising:
a. concentrating extracellular vesicles in a conditioned medium with a tangential flow filter;
b. determining a concentration of the extracellular vesicles to be at least 5×10¹⁰ extracellular vesicles/mL;
c. contacting the extracellular vesicles with a green fluorescent RNA stain or a red fluorescent RNA stain;
d. incubating the contacted extracellular vesicles to generate a labeled extracellular vesicle population;
e. diluting the labeled extracellular vesicle population by at least a factor of 1:300;
f. recovering the labeled extracellular vesicle population; and
g. analyzing the recovered, labeled extracellular vesicle population using a flow cytometer for nanoparticle analysis,
wherein the extracellular vesicles are not purified via size exclusion chromatography prior to the contacting in c.
